(19) United States
(12) Patent Application Publication
(10) Pub. No.: US 2009/0248034 A1
(43) Pub. Date: Oct. 1, 2009

(54) Method of Diagnosing and Treating Benign Prostatic Hyperplasia

(75) Inventors: Mark J. Dolan, Santa Rosa, CA (US); Dwayne S. Yamasaki, St. Augustine, FL (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/058,168

(22) Filed: Mar. 28, 2008

(51) Int. Cl. A61B 17/221; A61B 6/00; A61F 2/82; A61M 36/00; A61M 29/02; A61B 17/22

(52) U.S. Cl. 606/128; 600/431; 623/1.2; 623/1.1; 623/1.42; 606/159; 606/194; 606/200; 600/3; 128/898

(57) ABSTRACT

A method of diagnosing and treating a patient having benign prostatic hyperplasia caused by insufficient blood flow to the prostate due to atherosclerosis of a pelvic artery is disclosed. A method of diagnosing the patient's condition includes determining if a stenosis exists within a pelvic vessel. A method of treating the patient's condition may include placing a stent within the stenosed pelvic artery.

### FIELD OF THE INVENTION

The present invention relates to a method of diagnosing and treating benign prostatic hyperplasia. More particularly, a method of diagnosing a patient having benign prostatic hyperplasia caused by atherosclerosis into or through the internal pudendal, inferior vesical, and/or middle rectal arteries for treatment by placement of a stent within the stenosed region of the affected vessels.

### BACKGROUND OF THE INVENTION

The prostate is a walnut sized gland found beneath the bladder and in front of the rectum that surrounds part of the male urethra. The prostate goes through two main periods of growth. In early puberty, the prostate doubles in size, and then, around age 25, the prostate begins to grow again and continues to grow throughout most of a man's life. The continuing enlargement of the prostate does not usually cause problems until later in life. However, the second period of growth may, many years later, result in Benign Prostatic Hyperplasia or BPH.

Hyperplasia is a general term for an increase in the number of the cells of an organ or tissue causing it to increase in size. It may be due to any number of causes including, but not limited to, increased demand, chronic inflammatory response, hormonal dysfunctions, or neoplasia. The exact cause of BPH is not known, though it is thought to be related to aging and changing hormone levels associated therewith.

According to the National Institute of Diabetes and Digestive and Kidney Diseases, BPH rarely causes symptoms in men before age 40, whereas more than half of men in their 60s and as many as 90 percent of men in their 70s and 80s have some symptoms of BPH. As the prostate enlarges, it presses against the urethra and interferes with urination. At the same time, the bladder wall becomes thicker and irritated, and begins to contract even when it contains small amounts of urine, which causes more frequent urination. Further, as the bladder continues to weaken, it may not empty completely and leave some urine behind. Blocking or narrowing of the urethra by the prostate and partial emptying of the bladder cause many of the problems associated with BPH.

Symptoms of BPH include blood in the urine, dribbling after voiding, feeling the bladder has not fully emptied after urination, a hesitant, interrupted or weak urine stream, pushing or straining to begin urination, and/or recurrent sudden urges to urinate. Diagnosing BPH in its earlier stages can lower the risk of developing complications, as delay may cause permanent bladder damage for which BPH treatment may be ineffective. In addition to a complete medical history and physical examination, diagnostic procedures for BPH may include: a digital rectal exam (DRE), which is a procedure in which the physician inserts a gloved finger into the rectum to examine the rectum and the prostate gland for signs of cancer; a renal ultrasound to determine the size and shape of the kidney and to detect a mass, kidney stone, cyst, or other obstruction or abnormalities; intravenous pyelogram (IVP), which is a series of x-rays of the kidney, ureters, and bladder with the injection of a contrast dye into the vein, to detect tumors, abnormalities, kidney stones, or any obstructions, as well as to assess renal blood flow; a cystoscopy, also known as cystourethroscopy, which is an examination in which a scope is inserted through the urethra to examine the bladder and urinary tract for structural abnormalities or obstructions, such as tumors or stones; and/or a urine flow study, which is a test in which the patient urinates into a special device that measures how quickly the urine is flowing, wherein a reduced flow may suggest BPH.

Treatments for BPH range from observation to medical treatment to surgery. If warranted, surgery to remove only the enlarged tissue that is pressing against the urethra is performed with the remainder of the prostate being left intact. Types of prostate surgery often include the following: transurethral surgery wherein no external incision is needed, as the surgeon reaches the prostate by inserting an instrument through the urethra, and transurethral resection of the prostate (TURP), wherein a resectoscope or similar instrument is inserted through the penis to perform a resection of the prostate. Another option is a transurethral incision of the prostate (TUIP), which is a procedure that widens the urethra by making some small cuts in the bladder neck at the location where the urethra joins the bladder and in the prostate gland itself. The use of a laser in performing a laser prostatectomy to vaporize obstructing prostate tissue is also an option. When the prostate gland is much enlarged and there are other complicating factors or when the bladder has been damaged and needs to be repaired, surgery that requires an external incision may be often required.

Non-surgical treatments may include: balloon urethroplasty wherein a thin tube with a balloon is inserted into the opening of the penis and guided to the narrowed portion of the urethra and inflated to widen the urethra and ease the flow of urine; transurethral microwave thermotherapy (TUMT), which employs a device called a Prostatron that uses microwaves to heat and destroy excess prostate tissue to reduce urinary frequency and urgency; and various medications, such as finasteride and alpha adrenergic blockers, that have been found to shrink or at least stop the growth of the prostate without using surgery.

In certain instances, the implantation of a prostatic stent within the narrowed area of the urethra may be medically necessary due to the extent of the urethral blockage and/or the age or health of the patient making him a poor surgical candidate. In such a procedure, the prostatic stent is tracked through the urethra to the narrowed or obstructed area and allowed to expand, to push back the prostatic tissue and widen the urethra. However the American Urological Association cautions that the placement of prostatic stents should be considered only in high-risk patients, for example, those with urinary retention, because prostatic stents are associated with significant complications, such as encrustation, infection and chronic pain.

Although there are many treatments and therapies for BPH, some cases of BPH may not be correctable or may be better addressed by a therapy other than those presently available. Accordingly, what is needed is a method of diagnosing and treating benign prostatic hyperplasia of an etiology not addressed by existing therapies.

### BRIEF SUMMARY OF THE INVENTION

An embodiment of the present invention is a method of diagnosing benign prostatic hyperplasia that is caused by insufficient blood flow to the prostate due to atherosclerosis of an artery that supplies blood to the prostate gland. In almost all cases, the artery is a pelvic artery, but there may be instances of other arteries that feed the prostate due to variations in the vascular anatomy. In a further embodiment, the artery is an internal pudendal, inferior vesical, and/or middle rectal artery.

Another embodiment of the present invention is a method of treating benign prostatic hyperplasia caused by atherosclerosis in a pelvic artery. The method includes tracking a stent delivery catheter to a stenosis within the pelvic artery and delivering a stent within the stenosis to restore flow through the pelvic artery. In an embodiment, the pelvic artery is one of the internal pudendal, inferior vesical, and middle rectal artery that is reached by tracking the stent delivery catheter through the femoral artery, the internal iliac artery and then into one of the internal pudendal, inferior vesical, and middle rectal artery to the site of the stenosis.

In various other embodiments of diagnosis and treatment of a pelvic artery in accordance with the present invention, the pelvic artery may be one of the internal pudendal, inferior vesical, and/or middle rectal arteries. In addition to stenting, various treatments for revascularization of a pelvic artery in accordance with embodiments of the present invention include angioplasty, rotational atherectomy, ultrasonic or other vibrational mechanisms to break-up the stenosis with or without aspiration, intravascular brachytherapy, stenotic masceration such as by angiojets, clot retrieval, and/or drug/biologic delivery.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an illustration of a guide catheter and stent delivery system for use in a method of treating benign prostatic hyperplasia according to an embodiment of the present invention.

### DETAILED DESCRIPTION OF EMBODIMENTS OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

Benign prostatic hyperplasia is a disease that has several etiologies, one of which may be insufficient blood flow through the pelvic arteries that feed the prostate gland. The internal pudendal, inferior vesical, and/or middle rectal arteries are branches of the internal iliac artery, also called the hypogastric artery, which is a branch of the common iliac arteries. The arteries of the prostate are derived from the internal pudendal, inferior vesicle, and middle rectal arteries.

Atherosclerosis occurring anywhere along the arterial path from the abdominal aorta through the internal pudendal artery can adversely affect blood flow to the prostate arteries. Chronic ischemia is known to lead to stromal fibrosis, glandular cystic atrophy and impaired smooth muscle relaxation, wherein ischemia-induced structural tissue damage has been associated with decreased vascular endothelial growth factor expression. In addition, impairment of neurogenic relaxation appears to involve changes in the nitric oxide pathway. Together or separately these changes may result in an alteration of mechanical properties of the prostate, with a loss of elasticity and an increase in smooth muscle tone. Overall stiffness of the prostate likely ensues with a resultant increase in resistance to urinary flow.

As such, stenting within the pelvic arterial region may be effective in treating benign prostatic hyperplasia caused by the vascular disease by increasing blood flow to the prostate, which may improve or restore balance in stomal, glandular and/or vascular endothial growth factor expression and/or restore production and/or delivery of nitric oxide to the prostate. In addition, an increase in blood flow to the prostate may improve the flushing of metabolites and the delivery of hormones to the gland. The improved delivery of hormones to the prostate may be especially beneficial, as a natural decrease in hormone levels occurs during the aging process making adequate circulation of the limited hormones remaining essential in maintaining or regaining prostate health.

In accordance with an embodiment of the present invention, a patient first undergoes a diagnostic procedure in order to determine whether a revascularization procedure is likely to alleviate benign prostatic hyperplasia in the patient. An initial step in making the diagnosis is to perform a pelvic angiogram on the patient. An angiogram is a diagnostic procedure using an x-ray to visualize blood vessels by following the introduction of a contrast material through an artery or system of vessels. A pelvic angiogram showing a stenosis in one of the pelvic arteries, such as the internal pudendal, inferior vesical or middle rectal arteries, may be treatable with a stent or other revascularization procedure to increase blood flow to the prostate arteries. In another embodiment of the present invention, other diagnostic procedures, such as a CT scan, MRI, CT-angiography (CTA), MR-angiography (MRA), indirectly via doppler or color duplex sonography, and nuclear imaging, i.e., radionuclide angiography, may be used to determine whether a stenosis exists in one of the pelvic arteries.

Upon diagnosis of a stentable or otherwise treatable stenosis within one of the patient's pelvic arteries, such as the internal pudendal, treatment of the patient's benign prostatic hyperplasia may be effectuated by placement of a stent. In accordance with various embodiments of the present invention, the stent may be one of a self-expanding stent or a balloon expandable stent that is placed within the stenosed region of the pelvic vasculature.

Due to the position of the internal pudendal artery within a man's anatomy, it may receive in vivo loading from forces external to the body that may potentially crush the internal pudendal artery, as well as any implanted stent therein, between the external force and the sacrospinous ligament. As such, if the stenosis to be treated is within the internal pudendal artery a self-expanding stent design may be warranted so that if subjected to acute high forces within the internal pudendal artery the self-expanding stent design will deform and then return to its original configuration. Thus in an embodiment of the present invention, a self-expanding stent may be implanted within the internal pudendal artery to provide vessel support within a stenosed region of the artery. The stent may be delivered to the treatment site by tracking a stent delivery catheter through an access site in the femoral artery then into the internal iliac artery and subsequently into the internal pudendal artery.

In addition, though contrary to conventional wisdom, recent testing performed by the inventors on a series of cadavers has proven that a balloon expandable stent design with sinusoidal elements was able to withstand direct external loading at the sacrospinous ligament and perineal loading directed at a balloon expanded stent implanted within the internal pudendal artery. Current balloon expanded stent designs would be desirable for use within the internal pudendal artery as, unlike self-expanding stents, they are readily available with outer expanded diameters that are small enough yet strong enough to be supportively implanted within the extremely narrow distal regions of the internal pudendal artery. Thus in another embodiment of the present invention, a balloon expandable stent may be implanted within the internal pudendal artery to provide vessel support within a stenosed region of the artery. The stent may be delivered to the treatment site by tracking a stent delivery catheter through an access site in the femoral artery then into the internal iliac artery and subsequently into the internal pudendal artery.

In various other embodiments of diagnosis and treatment of a pelvic artery in accordance with the present invention, the pelvic artery may be one of the common iliac artery, external iliac artery, and/or internal iliac or hypogastric artery. In addition to stenting, embodiments of the present invention include other procedures for revascularization of the pelvic artery. For instance, angioplasty, also known as balloon dilation or balloon angioplasty, is an effective therapy for some patients with artery disease and may be used to dilate or widen narrowed arteries. Balloon angioplasty utilizes a catheter with a deflated balloon on its tip for positioning within the narrowed part of the pelvic artery. Once properly positioned, the balloon is inflated and the narrowed area of the pelvic artery is widened to restore blood flow therethrough.

In another embodiment, a rotational atherectomy or rotoblading procedure may be preferred if the pelvic artery is blocked or occluded by a hard plaque. A rotational atherectomy may utilize a catheter with a high-speed burr, such as one coated with diamond tips, to grind the hard plaque inside the pelvic artery into fine particles that then pass downstream without blocking the patient's circulation. Alternatively, a distal protection device, such as a filter may be positioned downstream of the treatment site to trap embolic debris for subsequent removal.

In another embodiment where the stenosis is caused by a thromboembolic event, revascularization may include softening or breaking-up the stenosis or clot and then aspirating the particulate from the pelvic artery. The stenosis, whether a clot or obstruction, may be initially treated, i.e., loosened and/or broken up, through the use of an ultrasonic medical device, such as any of the devices disclosed in U.S. Pat. No. 6,660,013 to Rabiner et al. and U.S. Pat. No. 6,652,547 to Rabiner et al., each of which is assigned to OmniSonics Medical Technologies, Inc. of Wilmington, Mass., or by another mechanical disruption provided by, for e.g., sinusoidal wires, coils and the like, such as those available from EKOS Corporation of Bothell, Wash. A method in accordance with an embodiment of the present invention, includes tracking an ultrasonic guidewire or catheter to the obstruction within the lumen of an aspiration catheter and then activating the guidewire to break up the obstruction while the debris is aspirated through a catheter. Alternatively, initial treatment may include stenotic maceration by angiojets, such that the stenosis is adequately softened or thinned to be removed by a clot retrieval device or by aspiration.

In another embodiment, revascularization of the pelvic artery may include intravascular brachytherapy utilizing a catheter for delivering a radioactive source to the stenosis, such that the radioactive source may be placed within or proximate the vascular stenosis for radiation treatment.

In another embodiment, a drug-eluting stent or drug-eluting implant (hydrogel, endoluminal paving, polymeric tube, etc) can be implanted in the arteries feeding the prostate for local delivery of drugs to the prostate. Local drug delivery has the advantage of maximizing the dose to the target organ, while minimizing its effect systemically.

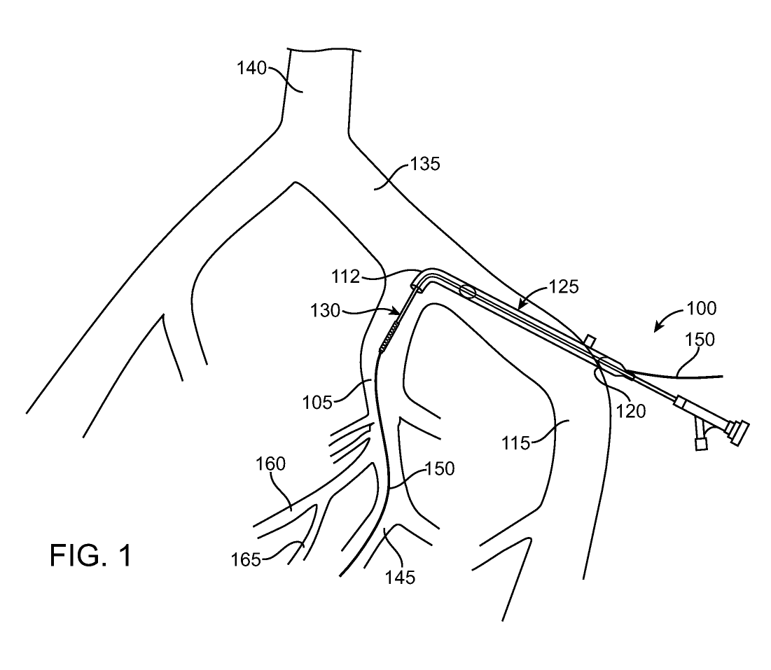

As shown in FIG. 1, a guide catheter and stent delivery system 100 may be used in embodiments of the present invention to more easily navigate the pelvic vasculature. System 100 is more fully disclosed in U.S. Patent Appl. Pub. No. US2006/0079951 A1 to Dolan et al., which is incorporated by reference herein in its entirety. System 100 is introduced into femoral artery 115 through a percutaneous access site 120 to be tracked over a guidewire 150, which in FIG. 1, extends within the pelvic vasculature to the treatment site (not shown) within the internal pudendal artery 145. System 100 includes a guide catheter portion 125 shown passing within femoral artery 115 and having an angled tip 112 directed into an ostium of internal iliac artery 105. A stent delivery portion 130 of system 100 is shown extending into internal iliac artery 105 from guide catheter portion 125. Stent delivery portion 130 is then maneuverable through internal iliac artery 105 into internal pudendal artery 145 to the site of the stenosis (not shown). For reference, the right common iliac artery and abdominal aorta are indicated at 135 and 140, respectively. In alternate embodiments, stent delivery portion 130 may be maneuvered through internal iliac artery 105 into one of the inferior vesical artery 160 or middle rectal artery 165 to the site of a stenosis (not shown) for placement of a stent. For an application as shown in FIG. 1, the length of guide catheter portion 125 of system 100 may be less than 50 centimeters.

A stent for use in embodiments of the present invention may be balloon expandable or self-expanding and may be made from any suitable medically implantable material, such as, but not limited to, stainless steel, nitinol, tantalum, ceramic, nickel, titanium, aluminum, polymeric materials, cobalt alloys, platinum iridium, titanium ASTM F63-83 Grade 1, niobium, high carat gold K 19-22, and combinations thereof. Stents and structures for stents suitable for use in embodiments of the present invention are disclosed in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated by reference herein in its entirety. As well, any suitable guide catheter and/or stent delivery catheter may be utilized in embodiments of the present invention, with or without a guidewire, as would be apparent to one of ordinary skill in the art.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A method of diagnosing benign prostatic hyperplasia correctable by arterial stenting, the method comprising:
identifying a stenosis in a pelvic artery that is restricting blood flow to the arteries of the prostate.

2. The method of claim 1, wherein the step of identifying a stenosis in a pelvic artery includes performing a pelvic angiogram.

3. A method of treating benign prostatic hyperplasia caused by atherosclerosis in an artery that supplies blood to a prostate gland, the method comprising:
tracking a delivery catheter having a stent to a stenosis within the artery; and
restoring blood flow through the artery by expanding the stent within the stenosis.

4. The method of claim 3 wherein the artery that supplies blood to the prostate gland is a pelvic artery.

5. The method of claim 4, wherein the pelvic artery is one of the internal iliac artery, external iliac artery, and common iliac artery.

6. The method of claim 4, wherein the pelvic artery is one of the internal pudendal artery, inferior vesical artery, and middle rectal artery.

7. The method of claim 3, wherein the stent used in the step of restoring blood flow through the artery is balloon expandable.

8. The method of claim 3, wherein the stent used in the step of restoring blood flow through the pelvic artery is self-expanding.

9. The method of claim 3, wherein the stent used in the step of restoring blood flow through the pelvic artery is a drug-eluting stent.

10. A method of treating benign prostatic hyperplasia caused by atherosclerosis in a pelvic artery, the method comprising:
tracking a revascularization tool to a stenosis within the pelvic artery; and
performing a revascularization procedure with the revascularization tool within the pelvic artery to substantially eliminate the stenosis and restore flow through the pelvic artery.

11. The method of claim 10, wherein the revascularization procedure includes rotoblading.

12. The method of claim 10, wherein the revascularization tool includes a vibrating portion for breaking-up the stenosis via mechanical vibration.

13. The method of claim 10, wherein the revascularization tool includes using angiojets for softening the stenosis for removal.

14. The method of claim 10, wherein the revascularization procedure is an intravascular brachytherapy and the revascularization tool includes a radiation source that is implantable within the stenosis.

15. The method of claim 10, wherein the revascularization tool includes a clot retrieval mechanism.

16. The method of claim 10, wherein the revascularization tool utilizes ultrasound for breaking-up the stenosis.

17. The method of claim 10, wherein the revascularization procedure is angioplasty and the revascularization tool includes a balloon catheter.
